(19) United States
(12) Patent Application Publication
(10) Pub. No.: US 2009/0196867 A1
(43) Pub. Date: Aug. 6, 2009

(54) SOY KEFIR POWDER AND USES THEREOF

(75) Inventor: Stan Kubow, Pointe-Claire (CA)

Correspondence Address: CHOATE, HALL & STEWART LLP, TWO INTERNATIONAL PLACE, BOSTON, MA, 02110, US

(73) Assignee: KCLM RESEARCH IN NUTRITION INC., Beaconsfield (CA)

(21) Appl. No.: 12/324,668

(22) Filed: Nov. 26, 2008

Related U.S. Application Data
(60) Provisional application No. 60/996,577, filed on Nov. 26, 2007.

(51) Int. Cl. A61K 38/02; A61P 25/00

(52) U.S. Cl. 424/116; 424/115

(57) ABSTRACT

The invention provides a method for treating fatigue and/or mood disorders and/or improving satiety and/or vitality in a subject in need thereof, comprising the step of administrating an effective amount of a soy kefir powder to said subject.

### CROSS-REFERENCE TO PRIOR APPLICATION

This application claims benefit of and priority to U.S. Provisional Patent Application Ser. No. 60/996,577, filed Nov. 26, 2007, the entire contents of which are herein incorporated by reference

### FIELD OF THE INVENTION

The present invention relates to kefir, and more particularly to soy kefir powder (SKP) used for treating fatigue, mood disorders, improving satiety and/or vitality.

### BRIEF DESCRIPTION OF THE PRIOR ART

Kefir originates from the Northern Caucasus Mountains where it has been consumed for centuries and has been valued for numerous health promoting properties.⁶ It continues to be a popular beverage in Eastern Europe, Scandinavia, and numerous individual countries^{13,14}. In the former Soviet Union, kefir has been traditionally used in hospitals and sanatoria for the treatment of numerous conditions including metabolic disorders, atherosclerosis, allergic disease, peptic ulcers, biliary tract diseases, chronic enteritis, bronchitis and pneumonia. It has also been used to treat tuberculosis, cancer, and gastrointestinal disorders when medical treatment was unavailable⁶.

Controlled clinical trials have yet to confirm the utility of most of the above clinical uses and currently there are no regulations on the sale of kefir or kefir extract as natural health products.

Kefir is a cultured milk beverage made by adding kefir grains to various milk products (i.e., cow, goat, soy, and other commonly consumed milks). The kefir grains ferment the milk, incorporating their probiotic organisms to create the cultured product. Kefir grains are not consumed as part of the final product; they are removed with a strainer at the completion of fermentation and added to a new batch of unfermented milk.

The kefir beverage has a tart, refreshing taste that is slightly acidic due to the presence of lactic acid. It is naturally effervescent due to the presence of carbon dioxide and minute concentrations of alcohol (i.e., 0.08% to 2%) as a result of yeast fermentation. Kefir also contains a variety of approximately 40 aromatic compounds, including diacetyl and acetaldehyde, which give it a characteristic flavour and aroma.¹

As the microbial composition varies significantly according to the kefir grain source, the source is critical to determining the final composition of the kefir product.^{2,3} The wide variety of microorganisms used in kefir fermentation differentiates kefir from virtually all other cultured milk products, which typically use only one and rarely more than three species in the culturing process.

Kefir grains are a soft, gelatinous white biomass, 3 to 20 mm in diameter, comprised of protein, lipids, and a water-soluble polysaccharide complex called Kefiran. Kefiran provides for a stable matrix that functions as a natural immobilized cell system. The grains resemble small cauliflower florets, their structure being the result of a symbiotic relationship shared between a large variety of specific lactic acid bacteria and yeasts. The grain matrix is composed of a complex of 13% protein (by dry weight), 24% polysaccharide, plus cellular debris and unknown components.²⁻¹²

Depending on the source of kefir grains, the microbial composition can vary significantly.²⁻⁶ The dominant microflora are Saccharomyces kefir, Lactobacillus caucasicus, Leuconnostoc species and lactic streptococci. Other probiotic microorganisms present in the grains include lactobacilli, such as Lb. acidophilus, Lb. brevis, Lb. casei Lb. casei subsp. rhamnosus, Lb. casei subsp. Pseudoplantarum, Lb. paracasei subsp. paracase, Lb. cellobiosus, Lb. delbrueckii subsp. bulgaricus, Lb. delbrueckii subsp. lactis, Lb. fructivoran, Lb. helveticus subsp. lactis, Lb. hilgardii, Lb. kefiri, Lb. kefuranofaciens, Lb. kefirgranum sp. nov, Lb. parakefir sp. nov, Lb. lactis, Lb. plantarum, Lb. cellobiosus and/or Lb. helveticus. Lactococci are also present such as subspecies of Lc. lactis, Lc. lactis var. diacetylactis and/or Lc. lactis subsp. Cremoris. Leuconostoc mesenteroides, Leuconostoc cremoris and L. cremoris are also present. Other bacteria include Streptococci salivarius subsp. thermophilus, and/or S. lactis, Enterococcus durans. Other bacteria include Acetobacter aceti and/or A. rasens.²⁻⁸

Bacteriocin may also be present, especially if the appropriate strains of lactic acid bacteria are present in the grains.^{2,9}

Varieties of yeasts such as Kluyveromyces, Candida, Torulopsis, and Saccharomyces sp. are also present in kefir grains. Candida albicans has not been found in kefir grains. Certain yeasts of kefir include the name Candida as part of their nomenclature. These kefir yeasts are not opportunistic yeasts such as C. albicans, but are classified as Generally Regarded As Safe (GRAS). Such yeasts may have the potential to keep C. albicans under control in the host.

The mean ranges of unit counts of microbes in gram stained kefir grains are, a) bacilli, 62-69%, b) streptococci 11-12%, and c) yeast, 16-20%.

Extracts of fermented soy foods have angiotensin converting enzyme (ACE) inhibitory and blood pressure (BP) lowering properties comparable to those of ACE inhibitor drugs.³³ Soy hydrolysates and soy ACE inhibitory peptides have been demonstrated to inhibit ACE activity in vascular tissue and to lower systolic blood pressure (SBP) in spontaneously hypertensive rats.³³⁻³⁵ Moreover, anti-hypertensive effects have been obtained from milk fermented with a combination of various lactic acid bacteria and yeast, a process analogous to kefir fermentation, albeit that kefir grains contain a greater variety of bacteria and yeast.²⁷

Chronic fatigue syndrome has been associated with higher serum angiotensin-converting enzyme (ACE) levels, which has been suggested to reflect damage to the vascular endothelium (Lieberman and Bell, 1993). Hence, a part of the efficacy of SKP in chronic fatigue might be related to its demonstrated ACE inhibitory activity.

Yeast fermentation generates higher levels of metabolite hydroxymethylbutyrate from the amino acid leucine, which promotes greater gains of muscle mass and strength in untrained men and women initiating resistance training (Nissen et al., 1996). Since yeasts are predominant microbiological species involved in kefir fermentation, kefir fermentation likely greatly increases the production of hydroxymethylbutyrate.

Several supplementation trials have indicated that branched chain amino acids (BCAA) can contribute to combat fatigue and to improve mental and physical performance in athletes (De Lorenzo et al., 2003; Blomstrand et al., 1991, 1997). Soy protein is one of the best sources of BCAA and fermentation increases the quantity of soluble proteins. Hence, the digestibility and bioavailability of BCAA from soy kefir would be significantly enhanced to provide a significant enhancement in BCAA uptake. An imbalance in the ratio of free tryptophan to BCAA with relatively low blood levels of BCAA has been implicated as a possible cause of acute physiological and psychological fatigue (central fatigue) during exercise (Gastmann and Lehmann, 1998) and the chronic fatigue syndrome (Georgiades et al., 2003).

The improvement of vitality has been sparsely investigated with no reference to fermented soy products. Green tea and products containing green tea, which contain caffeine, have been indicated to increase vitality or have stimulatory effects (Dulloo et al., 1999; Boon et al., 2004).

Other putative bioactive ingredients in soy kefir are isoflavones. Soybeans contain the highest natural concentration of isoflavones of any food.⁴³ The major dietary isoflavones found in soy are genistein, daidzein, formononetin, biochanin A and coumestrol. The biologically active isoflavones, genistein and daidzein, are substantially increased with soy protein fermentation.⁴⁴

Soy phytoestrogens have been shown to influence learning and memory (Lund et al., 2001a), affect aggressive and social behavior (Simon et al., 2004) and produce anxiolytic effects (Lund et al., 2001b).

In studies of rats, soy protein has been shown to have short-term satiating properties when compared to other sources of protein (Semon et al., 1987).

However, the presence of yeast protein as found in soy kefir powder and protein fermentation likely greatly increases satiety effects. For example, mycoprotein produced by the continuous fermentation by mushroom microorganisms induces acute and delayed suppressive effects on food intake (Turnbull et al., 1993). Also, rats fed a high yeast protein load reduced their meal and daily energy intake to a signifinicanty greater extent than rats fed well-balanced, high protein diets from a variety of protein sources including soy, total milk protein, or wheat gluten (Faipoux et al., 2006). The strong satiety may have been increased by protein hydrolysis for greater availability of absorption and/or gastrointestinal action of resulting peptides.

Fermentation of food proteins increases their digestibility and allows for greater absorption of peptides, without changing the overall biological value.⁷⁰ In particular, proteins with high disulfide content such as soy are relatively resistant to digestion,⁷¹ and fermentation increases their digestibility to allow for greater absorption of peptides.⁷⁰⁻⁷² We postulate that some physiologically active bioactive peptides may be present in their inactive forms in the amino acid sequences of proteins and are normally poorly absorbed from undigested soy proteins.

Fermentation may release these “hidden” peptides and subsequently exert health benefits. Small dipeptides and tripeptides, and even large peptides (10-51 amino acids) can be absorbed intact through the intestines and produce biological effects.⁷³⁻⁷⁴ It is noteworthy that ACE inhibitory peptides derived from milk fermentation have been shown to be resistant to the digestive condition and to exert a BP lowering effects when given orally to spontaneously hypertensive rats.⁷⁵ Isoflavonoids undergo acidic and enzymatic hydrolysis in the human gut and the isoflavones, biochanin A and formononetin, undergo demethylation to yield the aglycones genistein and daidzein, respectively. This metabolism may vary among individuals, resulting in differences in the relative proportions of isoflavonoid metabolites produced in the gut.⁷⁶

The half-lives of isoflavones are about 4-8 h, which suggests that maintenance of high plasma concentrations of isoflavone metabolites could be achieved with regular and frequent consumption of soy products.⁷⁷

For centuries, Asians have consumed fermented soy products with ACE inhibitory activity such as soy sauce and natto,^{78,79} with no documented adverse effects being noted apart from an adverse drug-food interaction noted with monoamine oxidase inhibitor drugs.^{80,81} While the presence of isoflavones with putative hormonal like activities (i.e., genistein and daidzein) may cause some safety concern, a review of the literature indicates that 40 g of soy powder contains 6-23.2 mg daidzein and 0.076-33.6 mg genistein. A typical 60 kg person consuming 40 g soy powder/day will not be exposed to more than 0.39 mg/kg/day daidzein or 0.56 mg/kg/day genistein. Animal studies, while limited, demonstrate that adverse effects were only observed at levels of isoflavones that are at least approximately 100 times higher than that found in 40 g of soy powder.

A comparison of soy kefir findings with other clinical trials using sf-36 health survey scores is described in B. Stacey, B. Parsons, S. Huang, S. Peyser and E. Dukes (2004) Gabapentin and Improved Health Status in Elderly Patients with Postherpetic Neuralgia: A Pooled Analysis of Three Clinical Studies. P&T 29: 646-651.

The Kadian® product monograph©2005 Alpharma Branded Products Division Inc. (KAD-CLMON-01 January 2005) (KADIAN sustained-release capsules contain Morphine Sulfate) is described in Reg Anesth Pain Med 2004; 29(2):A32.

### SUMMARY

The present invention provides a method for treating fatigue and/or mood disorders and/or improving satiety and/or vitality in a subject in need thereof, comprising the step of administrating an effective amount of a soy kefir powder.

Accordingly, the soy kefir powder (SKP) contemplated by the present invention is obtained by fermentating soymilk with kefir grains from the Moscow kefir strain, and has at least one of the following characteristics:
a protein composition of approximately of 25-45% wet weight,
a carbohydrate composition of approximately of 5-45% wet weight,
a fat composition of approximately of 25-45% wet weight, or
an ash composition of approximately of 5-15% wet weight.

Accordingly, the soy kefir powder (SKP) contemplated by the invention is obtained by fermentating soymilk with kefir grains from the Moscow kefir strain, and has at least three of the following characteristics:
a protein composition of approximately of 25-45% wet weight,
a carbohydrate composition of approximately of 5-45% wet weight,
a fat composition of approximately of 25-45% wet weight, or
an ash composition of approximately of 5-15% wet weight.

Accordingly, the soy kefir powder (SKP) contemplated by the invention is obtained by fermentating soymilk with kefir grains from the Moscow kefir strain, and has the following characteristics:
a protein composition of approximately of 25-45% wet weight,
a carbohydrate composition of approximately of 5-45% wet weight,
a fat composition of approximately of 25-45% wet weight, and
an ash composition of approximately of 5-15% wet weight.

The present invention has one of the following advantages: it provides a soy kefir powder which has increased potency over related products derived from other processes. The soy kefir powder of the invention also has the advantage of being a natural product which may not cause side effects. The SKP of the invention may thus be safe to use by pregnant women or subjects under other medications. Moreover, the SKP of the invention is easily accessible to anyone as it may be obtained without the need of a prescription.

### BRIEF DESCRIPTION OF THE FIGURES

FIG. 1. represents the electrophoretic profile of fermented soymilk (having the highest peak) compared to unfermented soymilk.

FIG. 2 shows the differences in improvement of mean scores of SF-36v2 subscales at the endpoint versus baseline.
Five-point change in the SF-36v2 health status score is considered as “clinically meaningful” change (Frost M H, Bonomi A E, Ferrams C E et al, and the Clinical Significance Consensus Meeting Group. Patient, clinician, and population perspectives on determining the clinical significance of quality-of-life scores. MayoClin Proc 2002; 77:488-494; Samsa G. Edelman D, Rothman M L, et al. Determining clinically important differences in health status measures. Pharmacoeconomics 1999; 15:141-155; Rowbotham M C. What is a “clinically meaningful” reduction in pain? Pain 2001; 94: 131-132.
PF=Physical Funtioning; RP=Role Physical; GH=General Health; SF=Social Functioning; RE=Role Emotional; MH=Mental Health

FIG. 3. is a flow chart illustrating a method for preparing soy kefir powder according to a preferred embodiment of the invention.

FIG. 4 represents the clinical trial results for 20 g@60d; n=10.

FIG. 5 represents the clinical trial results for 20 g@30d; n=10.

FIG. 6 represents the clinical trial results for 30 g@60d; n=7 (A multivariate statistical analysis on the separate groups was undertaken).

FIG. 7 represents the clinical trial results for 30 g@30d; n=7. (A multivariate statistical analysis on the separate groups was undertaken).

FIG. 8 represents the clinical trial results for 30 g@60d; n=7. (A multivariate statistical analysis on the combined groups was undertaken).

FIG. 9 represents the clinical trial results for 30 g@30d; n=7. (A multivariate statistical analysis on the combined groups was undertaken).

### DETAILED DESCRIPTION OF THE INVENTION

### Definitions

By the term “Moscow Kefir grain” it is meant the kefir grain obtained under an exclusive licence from the All-Russia Dairy Institute (ARDI), 35 Lyusinovskaya Street, Moscow, Russia. Table 1 summarizes the composition of the microflora of the Moscow kefir grain.

As used herein, the term “treating” refers to a process by which the symptoms of a disorder are alleviated or completely eliminated. Thus, in the context of disorders caused by fatigue, mood disorders, and overeating, the symptoms are alleviated or completely eliminated.

The term “preventing” refers to a process by which the disorder is obstructed or delayed.

By the term “fatigue” is intended, for the purpose of this invention, a “lack of energy”, a “lack of vitality” or “weakness”, either short term or persistent, including symptoms of the chronic fatigue syndrome that involve unrefreshing sleep, after any exertion, weariness that lasts for more than a day, fatigue that is not the result of excessive work or exercise, fatigue substantially impairs a person's ability to function normally at home, at work, and in social occasions. Mild exercise often makes the symptoms, especially fatigue, much worse, sleep or rest does not relieve fatigue. Fatigue leads to physical symptoms that include sore throat, swollen lymph nodes in the neck or armpits, muscle pain, pain without redness or swelling in a number of joints, intense or changing patterns of headaches, short-term memory loss or a severe inability to concentrate that affects work, school, or other normal activities.

By the term “mood disorders”, is intended, for the purpose of this invention, disturbances in emotions that inhibit an individual from functioning well be it depression or mania.

By the term “overeating”, it refers to the consumption of an energy intake that is inappropriately large for a given energy expenditure, thus, leading to obesity in a subject.

By the term “vitality”, the invention refers to a healthy capacity for vigorous activity.

By the term “subject” is intended, for the purpose of this invention, any live form that is subject to fatigue, depressed mood, and overeating, including humans, farm animals, domestic animals, or zoological garden animals.

As used herein, the expression “an acceptable carrier” means a vehicle for containing a soy kefir powder of the invention that can be administered to a subject without adverse effects. Suitable carriers known in the art can be of any food format that can be mixed with powder. They include, but are not limited to, a liquid such as water, milk, juice or a solid such as cookies, nutrition bars, ice cream. Carriers may include a food product such as a yogourt.

By the term “soy kefir liquid” used in the examples, it is meant the liquid obtained by the fermentation of soymilk with the Moscow kefir grains.

In a composition, the % wet weight refers to g/100 g wet weight. For instance, a protein composition of 25% wet weight means that there are 25 g of protein in a composition of 100 g.

HRQOL means Health-Related Quality of Life.

### Soy Kefir Powder Contemplated by the Invention and Uses Thereof.

The present invention relates to the use of a soy kefir powder. Such a soy kefir powder may be obtained by fermentating soymilk with kefir grains from the Moscow kefir strain.

The composition of the Microflora of the Moscow Kefir Grains from the All-Russian Scientific Research Institute of Dairy Industry (ARDI) is shown in Table 1.

TABLE 1
Composition of the Microflora of the Kefir Grains from the All-Russian
Scientific Research Institute of Dairy Industry (ARDI)

| Microorganism | CFU/g | Species | Total Sum |
|---|---|---|---|
| Lactobacillus | 2.65 × 10⁸ (87.78)^{a} | Lactobacilli | 2.92 × 10⁸ |
| acidophilus* | | | (96.82) |
| Lactobacillus | 1.96 × 10⁶ (0.65) | Lactococci | 5.12 × 10⁶ |
| delbrueckii lactis | | | (1.64) |
| Lactobacillus kefiri | 2.5 × 10⁷ (8.28) | Yeasts | 4.78 × 10⁶ |
| (formerly L. brevis) | | | (1.53) |
| Lactobacillus | 4.00 × 10⁴ (0.01) | Total | 3.02 × 10⁸ |
| kefiranofaciens | | | |
| Leuconostoc | 1.80 × 10⁵ (0.06) | | |
| mesenteroides | | | |
| cermoris | | | |
| Lactococcus lactis | 1.7 × 10⁶ (0.91) | | |
| lactis | | | |
| Lactococcus lactis | 1.98 × 10⁶ (0.66) | | |
| cremoris | | | |
| Leuconostoc | 2.00 × 10⁵ (0.07) | | |
| mesenteroides | | | |
| mesenteroides | | | |
| Candida kefyr | 2.1 × 10⁶ (0.70) | | |
| Candida tenuis | 2.4 × 10⁵ (0.08) | | |
| Saccharomyces lactis | 1.26 × 10⁶ (0.42) | | |
| Saccharomyces | 1.18 × 10⁶ (0.39) | | |
| unisporus | | | |
| (Saccharomyces | | | |
| delbrueckii) | | | |

^{a}Data in parenthesis represent percentage of total microflora

The soy powder of the invention has at least one of the following characteristics:
a protein composition of approximately of 25-45% wet weight,
a carbohydrate composition of approximately of 5-45% wet weight,
a fat composition of approximately of 25-45% wet weight, or
an ash composition of approximately of 5-15% wet weight.

The % refers to g/100 g wet weight.

More specifically, the soy kefir powder contemplated by the present invention comprises the characteristics listed in the following Table 2:

| CONTROLS | SPECIFICATION | RESULTS | METHOD |
|---|---|---|---|
| Description | Fine Powder | Conform | Visual |
| Colour | Beige Powder | Conform | Visual |
| Odour | Slightly milk | Conform | Olfactive |
| pH | 4.0-4.5 | 4.22 | USP <791> |
| Protein | 29.4-44.2% | 36.8% | NPPF-143T |
| Fat | 28.6-42.8% | 35.7% | NPPF-083T |
| Carbohydrate | 9.6-14.4% | 12.0% | NPPF-083T |
| Loss on drying | 7.6-11.4% | 9.5% | USP <731> |
| Ash | 4.8-7.2% | 6.0% | USP <281> |
| Total Plate | — | 5.7 × 10⁶ org/g | USP <61> |
| Count | | | |
| Yeast & Molds | <1000 cfu/g | 50 org/g | USP <61> |
| Salmonella | Absent | Absent | USP <61> |
| spp. | | | |
| S. aureus | Absent | Absent | USP <61> |
| E. Coli | Absent | Absent | USP <61> |
| P. aeruginosa | Absent | Absent | USP <61> |
| Enterobacter | <100 cfu/g | Absent | MCB-240802 |
| spp. | | | |
| IC₅₀ | 2.6-4.0 mg/mL | 3.3 mg/mL | OPA-Chromogenic |
| | | | Reaction |

The method for preparing a soy kefir powder contemplated by the invention first comprises the step of fermenting soymilk with Moscow kefir grains under suitable time and temperature conditions to obtain a fermentation culture. The kefir grains are placed in a fermenter at a ratio, for instance, of 1 part grains to 40 parts soymilk (2% dextrose may be added), or at a ratio, for instance, of 1 to 20, or at a ratio, for instance, of 1 to 5. Then, the grains are fermented at room temperature for approximately 10 to 24 hours, preferably for 16 to 24 hours, and more preferably for 16 hours. As used herein, the term “room temperature” refers to a temperature value of about 23° C., for instance a temperature of 23° C.±5° C.

Prior to the step of fermenting soymilk with the kefir grain, kefir grains are activated in soymilk (2% dextrose may be added) for instance at a ratio of 1-part grains to 2-parts milk and maintained at room temperature for approximately 24 hours.

The method of the invention also comprises the step of separating the kefir grains from the fermentation culture. Indeed, as kefir grains increase in volume during fermentation, a portion of grains are removed to maintain constant grain-to-milk ratio. For instance, the grains are removed by coarse sieving and can be advantageously used as the inoculum for fermenting a subsequent batch of soymilk. Alternatively, grains may be lyophilized for long-term storage.

In order to remove any significant amount of alcohol, the method may further comprise a step of spray-drying the fermentation culture so as to obtain a soy kefir powder. The fermentation culture thus obtained may be spray-dried at a temperature of 65° C.±13° C. until a significant amount of alcohol is removed. Alternatively, the fermentation culture may be spray-dried at 180-250° C. instantaneously (<1 sec.). The kefir powder is then processed to separate from the same agglomerated kefir powder called “chunks”. In such a case, the chunks may be crushed and then added and mixed to the kefir powder.

### Current Status

Soy Kefir Powder is developed by the Applicant and contains live organisms, it is a probiotic; however, it is currently developed for its other properties. Using the Applicant's fermentation process, different peptide products are produced from soymilk vs. cow's milk, leading to important differences in the bioactivities of each product in cell culture; a greater bioactivity has been associated with soymilk based kefir. Therefore, the Applicant is focusing development efforts on fermented soymilk. The Applicant does not produce an actual kefir beverage, rather fermented soymilk is spray-dried and provided as a powder. The spray-drying process produces an alcohol free product. To consume, dry powder is preferably mixed with a suitable amount of liquid (i.e., water, juice, milk, etc.).

### Physical and Chemical Properties

Natural Health Product Substance

Appearance: Beige powder, similar in consistency to powdered milk.

Solubility: Soluble in water.

Manufacturer: The Applicant

Storage: Store refrigerated at 2-8° C.

Constituents:

Protein (approximate): 35-40%

Carbohydrate: 10-14%

Fat: 35-40%

Moisture: 8-10%

Ash: 5-7%

The % refers to g/100 g wet weight.

pH: 4.2

Stability: 12 months refrigerated at 2-8° C.

### Active Ingredients

The therapeutically active anti-hypertensive agents in soymilk fermented with kefir grains are postulated to be isoflavones.

Two main isoflavones in soybeans are genistein and daidzen with minor amounts of glycetein and glycetin. Isoflavones can exist in several different forms (glucoside, rnalonyl, acetyl, etc.) and these forms are interconvertible by enzymatic hydrolysis during fermentation or heat treatment. However, the total isoflavone content remains approximately the same. The presence of bacterial glucosidases during fermentation produces the aglycone form, genistein and daidzen, as the main products. Tempeh, miso and other fermented soy foods have lower isoflavone levels (0.36 to 1.38 mg/g) and higher aglycone levels than tofu due to the fermentation.

Soy Kefir Powder contains the following isoflavone content:

0.130 to 0.150 mg diazidin/g soy kefir powder=4.55 to 5.25 mg diazidin/35 g soy kefir powder

0.450 to 0.550 mg genistin/g soy kefir powder=15.75 to 19.25 mg genistin/35 g soy kefir powder

0.120 to 0.140 mg daidzein/g soy kefir powder=4.2 to 4.9 mg diazidin/35 g soy kefir powder

0.225 to 0.245 mg genistein/g soy kefir powder=7.875 to 8.575 mg genistein/35 g soy kefir powder.

Total isoflavone content for 35 mg=32.375 to 37.975 mg/35 g soy kefir powder. This amount of isoflavones corresponds to between 0.925 and 1.085 mg isoflavones/g soy kefir powder.

In another embodiment, Soy Kefir Powder contains the following isoflavone content:

0.149 mg diazidin/g soy kefir powder=5.215 mg diazidin/35 g soy kefir powder

0.492 mg genistin/g soy kefir powder=19.441 mg genistin/35 g soy kefir powder

0.129 mg daidzein/g soy kefir powder=4.52 mg diazidin/35 g soy kefir powder

0.235 mg genistein/g soy kefir powder=8.22 mg genistein/35 g soy kefir powder.

Total isoflavone content for 35 mg=37.40 mg/35 g soy kefir powder. This amount of isoflavones corresponds to 1.07 mg isoflavones/g soy kefir powder.

Isoflavone concentrations in foods are usually expressed on a per gram protein basis, since we previously have assessed the protein content of SKP to be 43%, the isoflavone concentration would be 2.49 mg/g protein, which is slightly above the range of isoflavones seen in the richest sources of isoflavones in soy products such as soy flour (approximately 2 mg/g protein). A study has shown that intake of 45 g soy flour leads to 50 to 100-fold increase in blood and urinary concentrations of isoflavones, respectively.

Studies using isoflavones have suggested that effective doses start about 30 mg isoflavones/day; however, it should be noted that the content of the more bioactive aglycone isoflavones genistein and diazidein is important clinically. It is interesting that there is a high proportion of daizidein and genistein, which is the most important biologically active isoflavone, in soy kefir powder. Investigations have shown that the isoflavone absorption is greater in humans consuming fermented soy products since fermented soy contains a high proportion of the better absorbed aglycone isoflavones (daidzein and genistein) than the glucose-conjugated forms (daizidin and genistin).

S-equol is a product of daidzin or daidzein biotransformation by intestinal bacteria. There is greater efficacy of soy protein or soy food diets in people that have an active bacterial flora capable of converting daidzein into S-equol. Using an in vitro model of the gastrointestinal tract, recent work has indicated that supplementation of equol-producing bacteria on daidzein metabolism can increase equol production and so it is possible that the probiotic effects of SKP bacteria may increase the potency of isoflavones in the product.

There is increasing evidence that isoflavones are ineffective when provided alone but require the presence of soy protein and other soy components to be effective such as lignans, proteins, etc.

### Assay Methodologies

Method for Determining Isoflavone Content

Analysis for isoflavone content of soy kefir powder was carried out as following:

Standard samples of the following isoflavones were used to make standard mixtures as following:

1—Daidzin 1.0 mg dissolved in 1.2 mL acetonitrile:H20:acetone (4:1:1)

2—Genistin 1.0 mg dissolved in 1.25 mL acetonitrile:H20 (4:1)

3—Daidzein 1.0 mg dissolved in 2.0 mL acetonitrile:H20 (9:1)

4—Genistein 1.0 mg dissolved in 1.25 mL acetonitrile:H20 (4:1)

5—Biochanin A 1.0 mg dissolved in 1.0 mL acetonitrile

6—In an Eppendorf vial, the following amounts of each standard solution were mixed to obtain a mixture which contains 100 μg of each isoflavone:
a. 120 μL Daidzin
b. 125 μL Genistin
c. 200 μL Daidzein
d. 100 μL Genistein
e. 100 μL Biochanin A
f. 350 μL acetonitrile

Using the 100 μg/mL solution mixture, a serial dilution was carried out to make standards mixtures of the following concentrations: 50, 25, and 12.5 μg/mL.

The standards mixtures were used to build the calibration curves.

The samples were extracted and analyzed as following:

1. 400 mg of each sample powder was suspended in 4 ml of acetonitrile:methanol (1:1) solution in 10 mL screwed cap tube. Each tube was subjected to sonication for 1 hour in ice cold water bath, with interval vortexing every 15 minutes.

2. Samples were allowed to settle down on bench for 15 min. Then the clear upper solution of each sample was transferred into 1.5 mL Eppendorf vial, and then centrifuged at 20000 G for 5 minutes.

The clear supernatant was further filtered through nylon filters (0.2 μm pores) and was analyzed by HPLC-UV.

### Use of the Soy Kefir Powder of the Invention for Improving Mood

An object of the invention relates to the use of the soy kefir powder as defined above for improving mood, and for preventing and/or treating mood disorders in a subject.

The Soy Kefir Powder of the invention is a Natural Health Product currently being developed for improving energy, mood and satiety. It is produced by the fermentation of soymilk using genuine Russian Kefir grains sourced from the All-Russia Dairy Institute. Milk is spray-dried following fermentation, resulting in a probiotic powder with a protein composition of approximately 35-40%.

Isoflavones, which are also found in more bioavailable forms in fermented soymilk, have been demonstrated to possess aniolytic properties, decrease aggressive behaviour, diminish the stress response and to lower incidence of depressive mood and improve cognitive performance in postmenopause. Therefore, the Soy Kefir Powder of the invention may provide multiple therapeutic modalities and be of potential benefit in numerous stress disorders.

In summary, the Soy Kefir Powder of the invention is a unique fermentation product that provides therapeutic benefits and improves the mood of subjects.

### Use of the Soy Kefir Powder of the Invention for Treating Fatigue

In another object, the present invention relates to the use of the soy kefir powder contemplated by the invention for preventing and/or treating fatigue.

The mechanisms of action for energy improvement with SKP are unclear but are likely multi-factorial. Chronic fatigue syndrome has been associated with higher serum angiotensin-converting enzyme (ACE) levels, which has been suggested to reflect damage to the vascular endothelium (Lieberman J and Bell, 1993). Hence, a part of the efficacy of SKP in chronic fatigue might be related to its demonstrated ACE inhibitory activity.

Yeast fermentation generates higher levels of metabolite hydroxymethylbutyrate from the amino acid leucine, which promotes greater gains of muscle mass and strength in untrained men and women initiating resistance training (Nissen et al., 1996). Since yeasts are predominant microbiological species involved in kefir fermentation, kefir fermentation likely greatly increases the production of hydroxymethylbutyrate.

Several supplementation trials have indicated that branched chain amino acids (BCAA) can contribute to combat fatigue and to improve mental and physical performance in athletes (De Lorenzo et al., 2003; Blomstrand et al., 1997). Soy protein is one of the best sources of BCAA and fermentation increases the quantity of soluble proteins. Hence, the digestibility and bioavailability of BCAA from soy kefir would be significantly enhanced to provide a significant enhancement in BCAA uptake. An imbalance in the ratio of free tryptophan to BCAA with relatively low blood levels of BCAA has been implicated as a possible cause of acute physiological and psychological fatigue (central fatigue) during exercise (Gastmann and Lehmann, 1998) and the chronic fatigue syndrome (Georgiades et al., 2003).

### Use of the Soy Kefir Powder of the Invention for Increasing Vitality

In another object, the present invention relates to the use of the soy kefir powder contemplated by the invention for increasing vitality.

The improvement of vitality has been sparsely investigated with no reference to any fermented soy products. Green tea and products containing green tea, which contain caffeine, have been indicated to increase vitality or have stimulatory effects.

The findings shown in Example 2 thus are unexpected showing that healthy individuals showed improvement in energy levels and vitality despite low daily intakes of soy kefir liquid (only 200 mL) or 25-35 g soy kefir powder, major improvements were noted in relatively quickly (i.e., within 1-7 days). As shown in FIG. 2, vitality was greatly improved in the clinical trial involving soy kefir powder, as a significant increase in the sub-scale measure of vitality in the SF-32v2 questionnaire scores was demonstrated with a 12.8 point increase over baseline measures. A five point increase in considered to be clinically meaningful. (Rowbotham, 2001).

### Use of the Soy Kefir Powder of the Invention for Improving Satiety

In a further object, the present invention relates to the use of the soy kefir powder contemplated by the invention improving satiety.

In studies of rats, soy protein has been shown to have short-term satiating properties when compared to other sources of protein (Semon et al., 1987).

However, the presence of yeast protein as found in soy kefir powder and protein fermentation increases greatly satiety effects. For example, mycoprotein produced by the continuous fermentation by mushroom microorganisms induces acute and delayed suppressive effects on food intake. Also, rats fed a high yeast protein load reduced their meal and daily energy intake to a signifinicanty greater extent than rats fed well-balanced, high protein diets from a variety of protein sources including soy, total milk protein, or wheat gluten (Faipoux et al., 2006). The strong satiety may have been increased by protein hydrolysis for greater availability of absorption and/or gastrointestinal action of resulting peptides.

Fermentation of food proteins increases their digestibility and allows for greater absorption of peptides, without changing the overall biological value.⁷⁰ In particular, proteins with high disulfide content such as soy are relatively resistant to digestion,⁷¹ and fermentation increases their digestibility to allow for greater absorption of peptides.⁷⁰⁻⁷² We postulate that some physiologically active bioactive peptides may be present in their inactive forms in the amino acid sequences of proteins and are normally poorly absorbed from undigested soy proteins.

The findings shown in Example 2 thus are unexpected showing that despite low daily intakes of soy kefir liquid (only 200 mL) or 25-35 g soy kefir powder, major satiety was noted in relatively quickly (i.e., within 1-7 days).

### Methods of Use

In related objects, the present invention provides methods for preventing and/or treating mood disorders, for treating fatigue, for increasing vitality, for improving satiety and/or preventing and/or treating fibromyalgia. Such methods comprise the step of administering to said subject an effective amount of the soy kefir powder of the invention.

The soy kefir powder may be associated with an acceptable carrier.

The amount of soy kefir powder of the invention administered is preferably an effective amount. An effective amount of soy kefir powder of the invention is that amount necessary to allow the same to perform its desired role without causing, overly negative effects in the subject to which the soy kefir powder is administered. The exact amount of soy kefir powder to be administered will vary according to factors such as the type of condition being treated, as well as the mode of administration. In the context of the present invention, is useful to administer an amount that will treat fatigue, mood disorders, improve satiety, improve energy and improve vitality.

The soy kefir powder of the invention may be given to a subject through various routes of administration, but it is however preferably administered per os. Suitable dosages will vary, depending upon factors such as the desired effect (short or long term), the route of administration, the age and the weight of the subject to be treated.

The present invention will be more readily understood by referring to the following examples. These examples are illustrative of the wide range of applicability of the present invention and is not intended to limit its scope. Modifications and variations can be made therein without departing from the spirit and scope of the invention. Although any methods and materials similar or equivalent to those described herein can be used in the practice for testing of the present invention, the preferred methods and materials are described.

### EXAMPLES

### Example 1

### Preparation of Soy Kefir Powder According to a Preferred Method of Preparation of the Invention

Hundred (100) cases of sterile (UHT) SO NICE Natural soy milk (Soyaworld Inc.), each case consisting of twelve 946 mL Tetrapaks, were obtained (three production lots). Milk was stored at 4° C. in a walk-in cold room.

A 150 L Chemap fermenter situated in the Biotechnology Research Institute (BRI, Montreal, Quebec) pilot plant was used for all production fermentations.

Prior to the first fermentation, the fermenter was cleaned using standard BRI protocols and then steam sterilized at 121° C. using the computer controlled sterilization cycle. The fermenter was equipped for on-line control of temperature and continuous monitoring of pH. The fermentations were run without air addition (anaerobic) and without agitation, except for brief periods during startup and harvesting.

The fermentation substrate consisted of soy milk and dextrose. Except for batches designated K0830A and K0830B, each batch used 9 cases of milk (102 L) plus 2 kg of dextrose. Batches K0830A and K0830B used 90 L of milk plus 1.78 kg of glucose. The temperature controller was set to a constant 23° C. The milk and glucose were added to the fermenter manually and then agitated for 2 minutes at 250 rpm prior to addition of the grains (starter culture). The initial starter culture consisted of 5 kg of wet grains plus 16 L of fermented kefir. In subsequent fermentations the grains consisted of the solids filtered from the previous batch of harvested kefir. After addition of the grains, the agitation was continued at 250 rpm for an additional 2 minutes. At this time the fermentation was considered as started and the pH and temperature were noted.

The fermentation was continued at constant temperature with no agitation for a specified length of time based on prior experience and ongoing analysis of the IC50 from previous batches. Except for the first batch, fermentation times ranged between 16 and 24 hours.

At the end of the batch and prior to harvesting, the kefir was agitated at 250 rpm for 2 min and the harvest line was flushed with steam. Since a pump was not used for harvesting, the kefir was removed from the fermenter by gravity flow, aided by 0.5 barr of air pressure introduced into the fermenter headspace.

The harvested kefir was filtered using a custom-made 316 SS cone sieve with 3.2 mm openings. The filtered liquid was collected in a 200 L SS tank and the collected solids retained for addition into the subsequent batch. From the SS tank, the filtered liquid was placed into 19 L plastic pails, sealed airtight with gasketed lids and placed in a walk-in cold room at 4° C. (note: this was a different cold room from that used for storage of the soy milk substrate).

The above-mentioned process can be repeated several times, for instance for a total of 11 fermentation batches.

After completion of all the fermentation batches, the liquid kefir was stored in a walk-in cold room at 4° C. Spray drying of the kefir was performed using a Niro Atomizer Spray Dryer Model HT-10-530. Each fermentation batch was spray dried separately. The spray drying conditions for each batch were maintained constant by controlling outlet air temperature to between 60-70° C. by adjustment of throughput rate. The time required and solids yield from each spray dried batch were recorded and, after obtaining a sample for analysis, each batch of powder was hermetically sealed in a plastic bag. After all batches had been spray dried, the powder from all batches (except K0817, first fermentation batch) was sieved using a Kason vibrating screen with 2.1 mm hole size. The total mass of large chunks collected by the sieving operation was 11.66 kg or about 23% of the total product yield. The large chunks were crushed using an Urschel high speed chopper and then added to the powder. All sieved and crushed powder was blended together for 30 minutes using a double-action ribbon blender. A 500 g sample was obtained for analysis.

The soy kefir powder was packaged in ten hermetically sealed plastic bags. Each bag was weighed and placed in an airtight plastic pail and stored at 4° C. until use.

### Example 2

### Use of the Soy Kefir Powder of the Invention for Treating Fatigue, Mood Elevation, and Increasing Satiety

### Raw Materials Used in Manufacturing

### Source of Soymilk

Soyaworld Inc., Burnaby, BC, Canada.

### Source of Kefir Grains

The All-Russia Dairy Institute (ARDI), 35 Lyusinovskaya Street, Moscow, Russia.

### Fermentation and Processing

Kefir grains are activated in unsweetened soymilk (2% dextrose added) at a ratio of 1-part grains to 2-parts milk and maintained at room temperature for approximately 24 hours. Grains are then removed by coarse sieving, placed in the fermenter at a ratio of 1 part grains to 5 parts soymilk (2% dextrose added), then fermented at room temperature for approximately 24 hours. Fermentation produces carbon dioxide gas and lowers the pH to 3.5 to 4, producing a thick foaming liquid, creamy in texture and consistency, with an approximate alcohol content of 0.5% to 1% by volume.

As kefir grains increase in volume during fermentation, a portion of grains are removed to maintain constant grain-to-milk ratio. When fermentation is completed, grains are removed by coarse sieving and used as the inoculum for fermenting a subsequent batch of soymilk.⁵ Alternatively, grains can be lyophilized for long-term storage.¹

Following removal of the grains, liquid kefir is approximately 8% total solids. It is then converted to powder by spray-drying, thus removing any significant amount of alcohol.

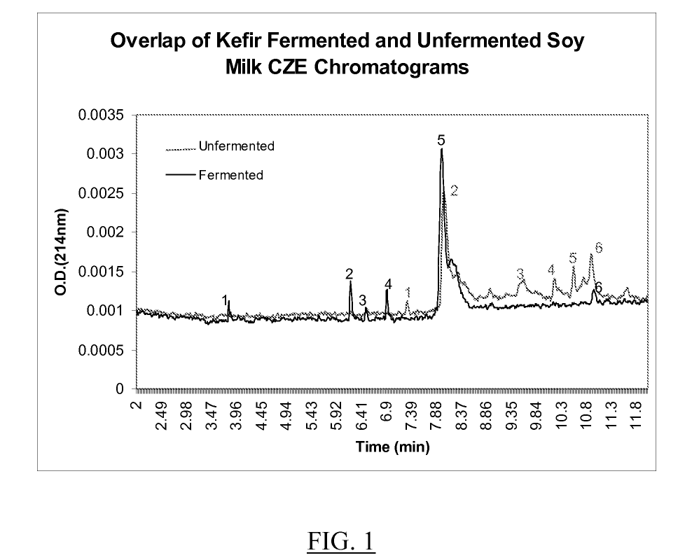

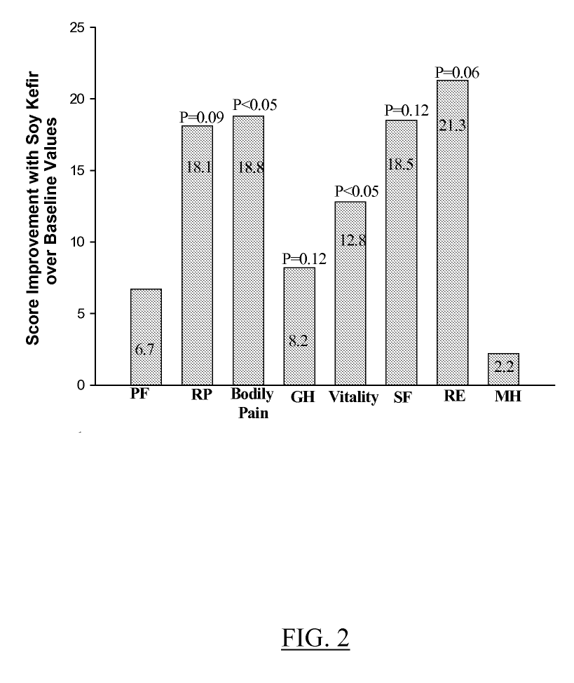

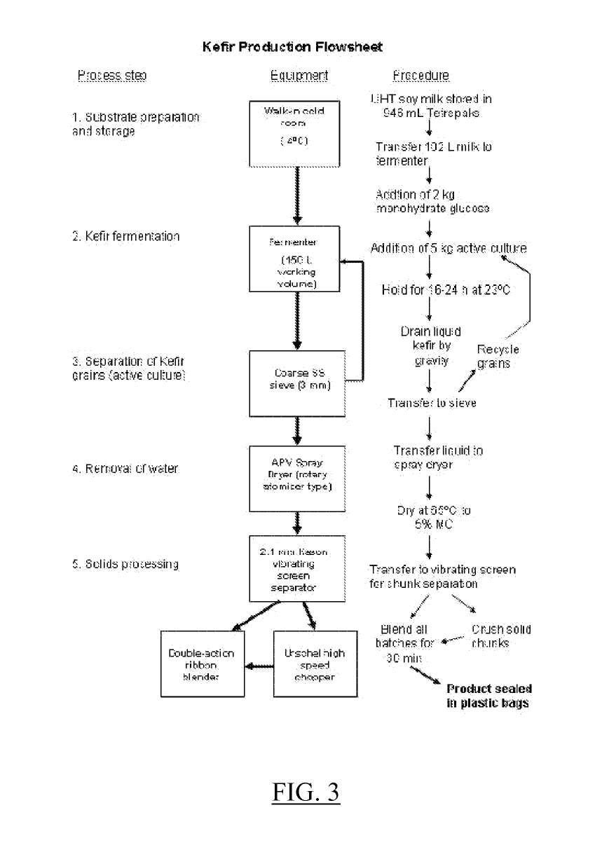

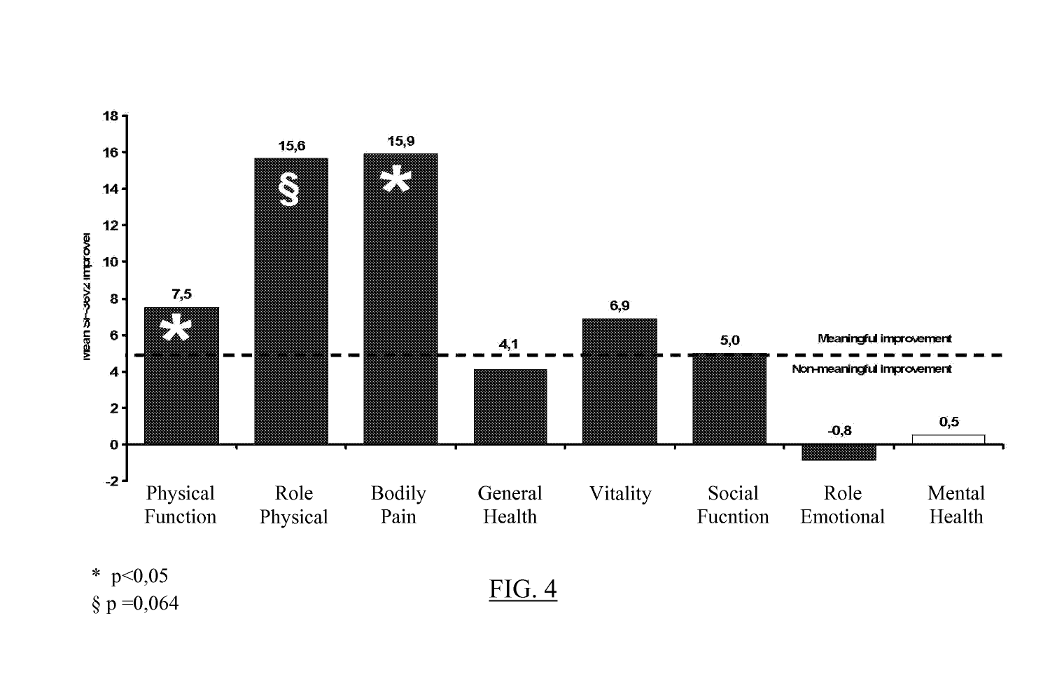

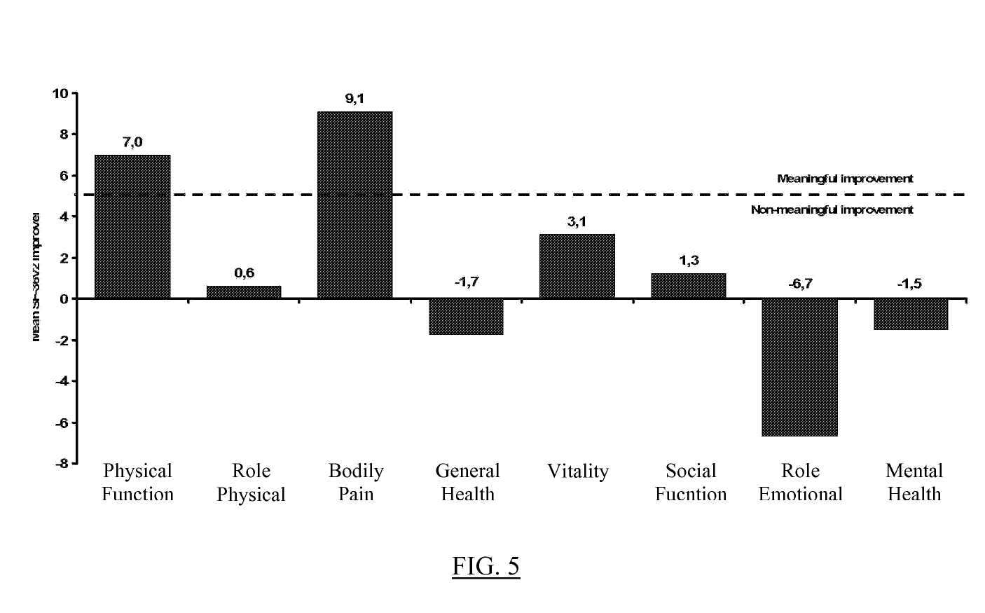

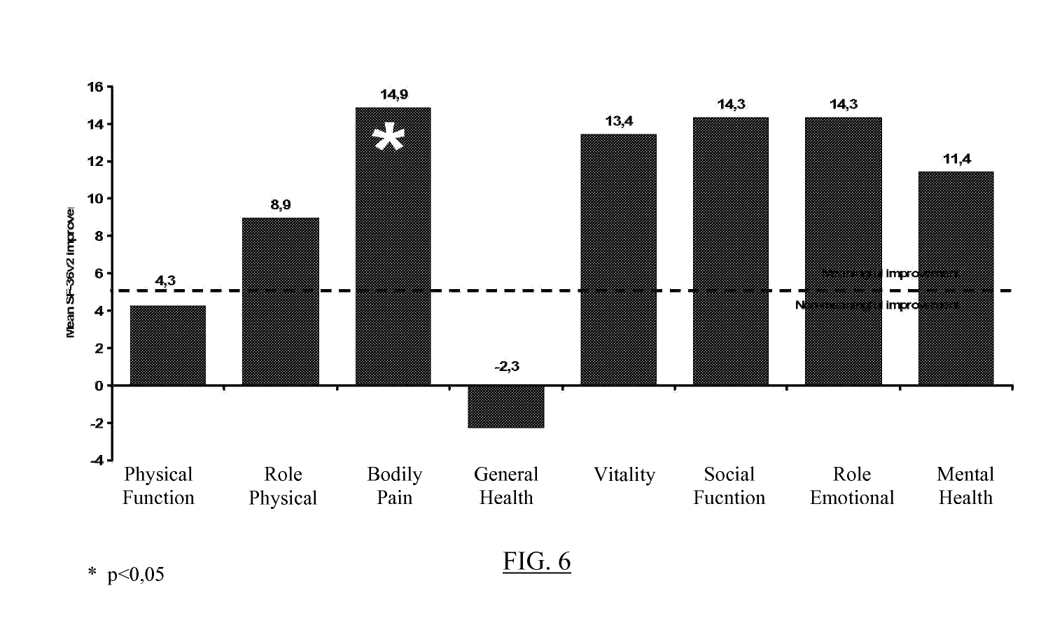

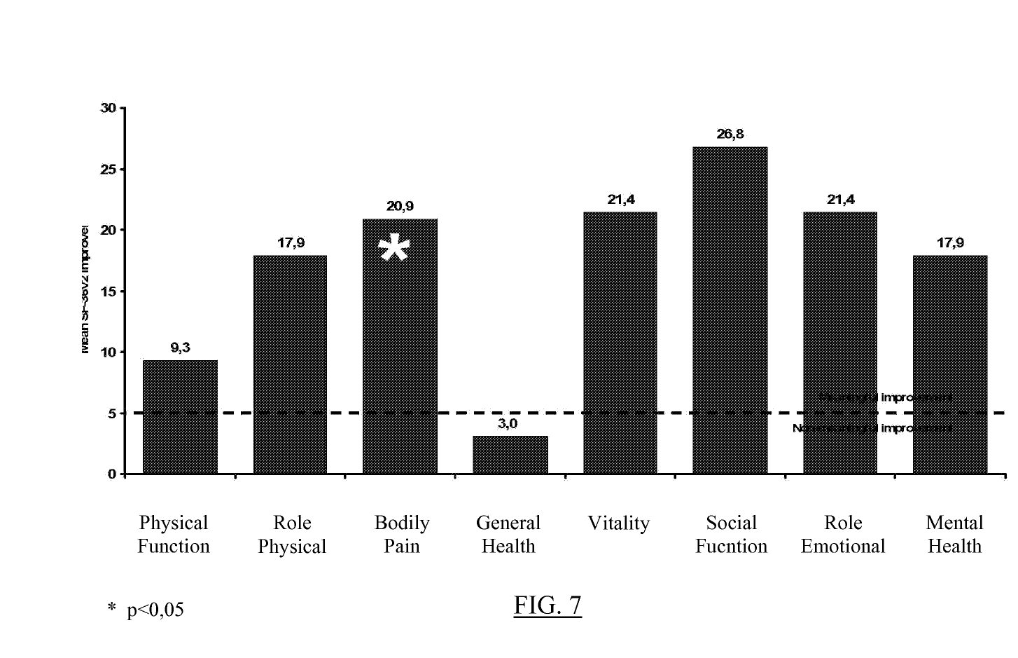

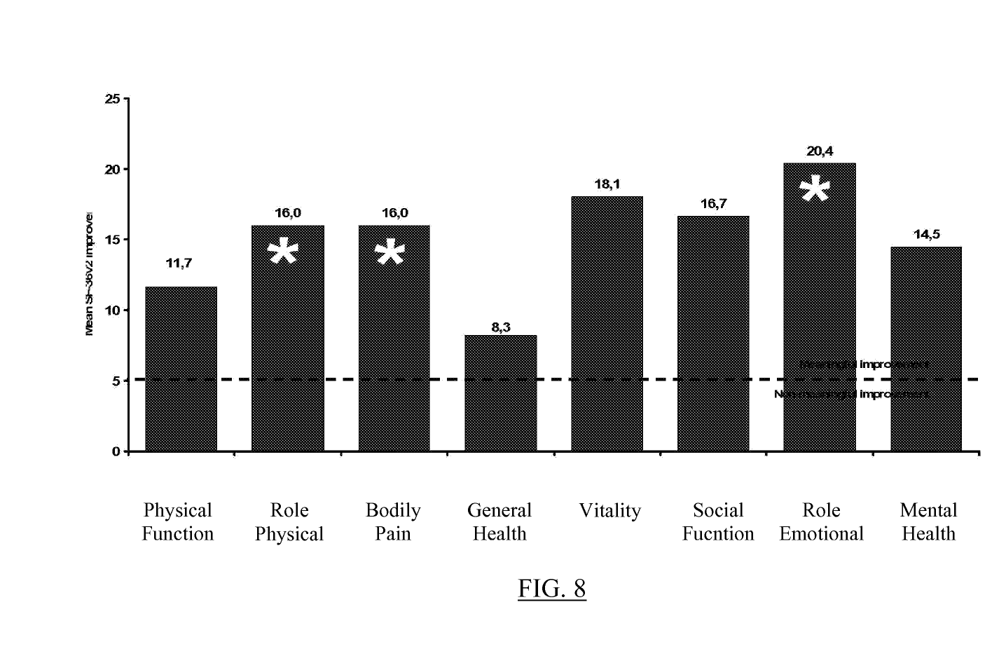

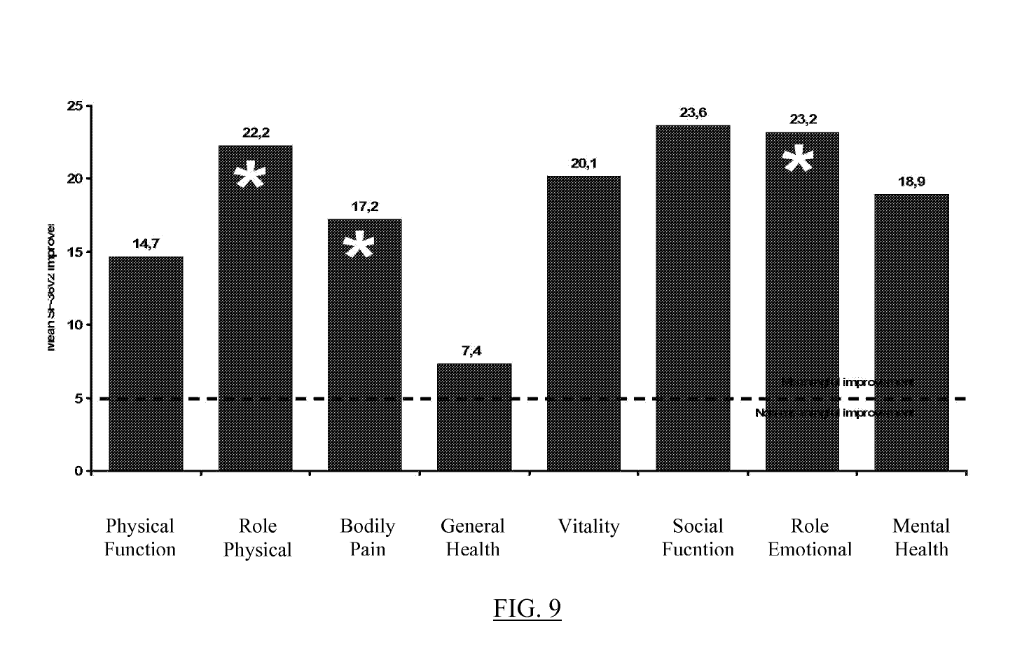

There are no significant variations of vitamin and mineral content following kefir fermentation from the original sourced soymilk; however, a small increase in proteolysis leads to an increase in free amino acids.²³ Indeed, the capillary electrophoretic profile of the fermented soymilk demonstrates a protein/peptide profile unique from that of unfermented soymilk (FIG. 1).

### Packaging

The soy kefir powder is packadged in 4½″×5½″ paper/foil pouches, each containing 35 grams of powder.

### Case Reports

Human data with Soy Kefir Powder or Soy Kefir Liquid shows significant effects in terms of mood elevation (i.e., stress reduction), relief of fatigue including the treatment of chronic fatigue syndrome in 10 subjects with and without chronic fatigue syndrome. Satiety effects were also specifically noted by some subjects.

Some subjects initially received the Soy Kefir Liquid only whereas others received Soy Kefir Liquid and SKP or SKP only. Subjects received initially the original batch of SKP (Trial 1) and at a later time point, most of the same subjects received the batch of SKP to be used in the clinical trial (Trial 2).

The first batch used generic Oriental store soy milk using a ratio of grains to milk of 1:5 whereas the second batch used SoNice soy milk using a ratio of grains to milk of 1:20.

For the batch of SKP produced for the clinical trial (Trial 2), symptoms regarding fatigue, stress/depression, sleep disturbances or other disturbances were rated on a scale 0-5 with a 0 being symptom-free and 5 meaning symptom at its greatest intensity. Any adverse event was also noted.

Open Label Clinical Trial with Patients with Chronic Fatigue Syndrome

As anecdotal evidence indicated that the Soy Kefir Powder may improve several features of the chronic fatigue syndrome, i.e., weakness, lack of energy and depressed mood, an open label pilot study was carried out to test the tolerance and effects of the product on a small group of chronic fatigue patients. Eleven patients with chronic fatigue syndrome received 56 pouches of 37.5 grams of product, to be taken as 1 pouch twice a day for 4 weeks. Patients answered the SF-36v2 Health Survey quality of life questionnaire before and after the 4 week treatment period.

The SF-36v2 Health Survey, is a highly validated, widely-used health status assessment tool that measures eight concepts: physical functioning (PF), role limitations due to physical health (RP), bodily pain (BP), general health perceptions (GH), vitality (VT), social functioning (SF), role limitations due to emotional problems (RE), and general mental health (MH). Scores for people at the top or bottom of a scale can be interpreted by looking at the items and response choices that must be chosen to earn those scores. For example, someone at the top score of the SF-36 Physical Functioning (PF) scale does not have limitations in any of the SF-36 activities due to health. A person scoring at the bottom of the PF scale is very limited in all activities, including bathing and dressing. Physical Functioning, Role Physical, and Bodily Pain are primarily measures of physical health, while the other three scales are primarily measures of mental health. Research has demonstrated that scales associated with the physical health construct are sensitive to detecting the impact of physical health interventions. Similarly, scales that are the strongest measures of mental health are sensitive in detecting the impact of mental health interventions. SF-36 is a FDA approved tool that is used in a wide variety of clinical areas ranging from cardiac rehabilitation programs and hip replacement surgery to the impact of medications on pain relief.

In the open label efficacy trial, two patients had to discontinue the treatment: one because of gastric pain after 3 days even though she said she had never felt so energetic from the time that she had the disease. She suggested that she wanted to try to take the product 1 or 2 days a week as her improvements were so remarkable. The second patient had gastric discomfort and vomited at her first ingestion of the product. Increased satiety effects were also noted after the ingestion of the product. All other patients took the product for 4 weeks. For the statistical analysis, a two-tailed Wilcoxon test was used. The results of the questionnaire are assembled into 8 scales and the average score for each scale before and after the treatment were compared. The alpha risk was 5%.

The results of this pilot study show that the product had significant beneficial effects on the subjects in terms of pain, energy and mood (FIG. 2). Two scales, Bodily Pain and Vitality show differences with an alpha risk <5% the accepted threshold in sciences and 4 other with an alpha risk ≦12% which qualify them for “trend” because they are compatible with a true effect (and consequently have a good chance to be shown in a larger study). Those additional scales that showed strong trends included Role Emotional, Role Physical, Social Functioning and General Health. A satiety effect was also noted in one subject in the pilot study, which was also observed in the case study among some subjects.

The clinical trial was extended from a two week to a four week intervention as there is evidence that the placebo effects typically fade after a two-week time frame. Hence, the placebo effect was therefore less likely as positive results were seen over the more extended period of four weeks. Moreover, literature indicates that the chronic fatigue patient population is quite resistant to placebo effects due to their high utilization of a wide variety of other products that show no efficacy. Also, improvement in the energy, emotional health and social functioning scores is concordant with the case study results showing consistent improvements in energy, mood elevation and satiety among subjects either with or without chronic fatigue syndrome.

### Example 3

### Case Studies of Individual Patients

Eleven patients experiencing different symptoms, some having chronic fatigue syndrome and the others suffering from other conditions were given soy kefir product individually at different times.

TABLE 3
The results of the clinicat study with patients having chronic fatigue syndrome:

| | | | |
|---|---|---|---|
| Therapeutic | | | Observations |
| Goal(s) | Dose | Treatment Duration | (therapeutic outcomes, adverse events, etc.) |

Subject TK (77, M)

| | | | |
|---|---|---|---|
| Increased energy | 200 ml QD (liquid) | Liquid (approx. 1 yr) | Consistently noted increased energy levels with soy kefir intake in each trial. |
| levels | 25 g QD (powder) | 25 g Powder | No adverse events were noted while receiving soy kefir (liquid or powder). |
| Satiety effects | 35 g QD (powder) | (2 mo - Trial 1) | |
| | | 35 g Powder (approx | |
| | | 1 wk - Trial 2) | |

Subject MP (46, F)

| | | | |
|---|---|---|---|
| Chronic fatigue | 35 g QD (powder) | 35 g Powder (approx 2 | Within 10 days of intake of clinical batch of SKP (Trial 2), subject showed |
| | | wk - Trial 2) | improvement in ratings related to fatigue going from 4.5 to 2 and stress going from 4.5 |
| | | | to 2.5. |
| | | | Subject also experienced a major improvement in energy levels that she had not |
| | | | experienced with any previous treatments. |
| | | | No adverse events were noted while receiving liquid soy kefir; no major drop in BP |
| | | | was noted with the intake of SKP. |

Subject WK (72 F)

| | | | |
|---|---|---|---|
| Increased energy | 200 ml QD (liquid) | Liquid (approx 1 yr) | Consistently noted increased energy levels with soy kefir intake in each trial. |
| levels | 25 g QD (powder) | 25 g Powder (2 mo - | No adverse events were noted while receiving soy kefir (liquid or powder). |
| Satiety effects | 35 g QD (powder) | Trial 1) | |
| | | 35 g Powder (approx | |
| | | 1 mo - Trial 2) | |

Subject PF (60, F)

| | | | |
|---|---|---|---|
| Mood Elevation | 35 g QD | 35 g QD (10 d) | Consistently noted mood elevation with soy kefir intake in each trial. |
| | (powder) | 17.5 g BID (10 d) | No adverse events noted. |
| | 17.5 g BID | | |
| | (powder) | | |

Subject DK (46, F)

| | | | |
|---|---|---|---|
| Increased energy | 200 ml QD (liquid) | Liquid (approx 4 mo) | Consistently noted increased energy levels and mood elevation with soy kefir intake |
| levels | 25 g QD (powder) | 25 g Powder (approx 4 | in each trial. |
| Mood elevation | 35 g QD (powder) | wk - Trial 1) | With intake of clinical batch (Trial 2), subject showed improvement in ratings related |
| | | 35 g Powder (approx | to fatigue and stress/depression from going from 4-5 to 2. |
| | | 2 wk - Trial 2) | No adverse events were noted while receiving soy kefir (liquid or powder). |

Subject NW (47, F)

| | | | |
|---|---|---|---|
| Chronic fatigue | 200 ml QD (liquid) | Liquid (approx 4 mo) | Consistently noted increased energy levels and mood elevation with soy kefir intake |
| Mood elevation | 25 g QD (powder) | 25 g per 2 days | in each trial. |
| Increased energy | 25 g per 2 days | Powder (approx 4 wk - | Relief of chronic fatigue noted within 1 week of intake of liquid or powdered soy kefir. |
| levels | (powder) | Trial 1) | Increased satiety was also noted. |
| Satiety effects | | 8 g per day (approx 4 | Each period of discontinuance of soymilk kefir was associated with a gradual |
| | | wk - Trial 2) | recurrence of chronic fatigue symptoms of depressed mood and decreased energy |
| | | | levels. |
| | | | This subject claims experiencing improvements in mood similar to that observed |
| | | | when taking serotonin re-uptake inhibitor drugs, that she received for chronic fatigue |
| | | | treatment |
| | | | Mood elevating and energy enhancing effects noted within 1 week of intake of liquid |
| | | | or soy kefir. |
| | | | To moderate the very high energy levels that were experienced, dosage in Trial 1 was |
| | | | lowered from 25 g QD to 25 g every other day. |
| | | | Similarly, in Trail 2, to moderate the very high energy levels, dosage of clinical batch |
| | | | of SKP (Trial 2) was lowered to 8 g per day. Subject showed improvement in ratings |
| | | | related to fatigue and stress/depression from going from 4-5 to 1. Subject |
| | | | experienced disturbed sleep due to increased energy levels at bedtime. |
| | | | No adverse events were noted while receiving soy kefir (liquid or powder). |

Subject EU (54, F)

| | | | |
|---|---|---|---|
| Mood elevation | 200 ml QD (liquid) | Liquid (2 mo) | Chronic depression and sleeplessness were relieved following intake of liquid soy |
| | | | kefir. |
| | | | No adverse events were noted while receiving liquid soy kefir. |

Subject RC (72, F)

| | | | |
|---|---|---|---|
| Mood elevation | 25 g QD (powder) | Powder (3 wk) | Chronic depression and sleeplessness were relieved following intake of soy kefir. |
| | | | History of chronic heartburn requiring medication (Nexium). Intake of soy kefir was |
| | | | associated with an aggravation of a long-standing and chronic heartburn condition for |
| | | | which the subject was taking regularly taking Nexium, which led the subject to |
| | | | discontinue soy kefir intake. No other adverse events were noted while receiving |
| | | | liquid soy kefir. |

Subject JB (F)

| | | | |
|---|---|---|---|
| Mood elevation | 25 g QD (powder) | Powder (2 wk) | Chronic depression was relieved and increased energy was noted following intake of |
| Increased energy | | | soy kefir. |
| levels | | | No adverse events were noted while receiving liquid soy kefir. |

Subject SK (47, M)

| | | | |
|---|---|---|---|
| Increased energy | 200 ml QD (liquid) | Liquid (approx 18 mo) | Consistently noted increased energy levels with soy kefir intake in each trial. |
| levels | 25 g QD (powder) | 25 g Powder (2 wk - | No adverse events were noted while receiving soy kefir (liquid or powder). |
| Satiety effects | | Trial 1) | |

Subject AC (F, 52)

| | | | |
|---|---|---|---|
| Increased energy | 200 ml QD (liquid) | Liquid (approx 18 mo) | Consistently noted increased energy levels and mood elevation with soy kefir intake |
| levels | 25 g QD (powder) | Powder (approx 2 wk - | in each trial. |
| Mood elevation | | Trial 1) | No adverse events were noted while receiving soy kefir (liquid or powder). |
| Satiety effects | | | |

### Example 4

### Protocol Outline

<<Effects of Soy Kefir Powder in Mild to Moderate on the Quality of Life of Patients with Chronic Fatigue Syndrome”

### Introduction:

From anecdotic evidence, it was hypothesized that a fermented soy milk product may improve several features of the chronic fatigue syndrome: asthenia, pain, and mood. A pilot study was planned to test the tolerance and effects of the product on a small group of patients and with an open design.

### Patients and Procedures:

Eleven patients with chronic fatigue syndrome received 56 pouches of 37, 5 gr of product, to be taken as 1 pouch twice a day for 4 weeks. Patients answered a quality of life questionnaire before and after the 4 week treatment period. This questionnaire, the SF36v2 is a validated tool that is widely used in this area of research.

### Results:

Tolerance: 2 patients had to discontinue the treatment: one because of gastric pain after 3 days even though she said she had not felt so energetic since she had the disease. She suggested trying to take the product 1 or 2 days a week. The second patient had gastric discomfort and vomited at her first ingestion of the product. All other patients took the product for 4 weeks.

Anecdotic findings: one patient noticed that after the ingestion of the product at around 10 am and 3 pm, she felt “full’ and did not experience hunger at the time of the next meal. Several patients had problems with the taste and did not find a perfect vehicle for the product. One of them did a thorough investigation of different beverages and found the brand of fruit juices CERES was the best.

One patient said that she had suffered from chronic muscle pain in here thighs and that it disappeared soon after the beginning of the treatment.

III Statistical analysis: the questionnaire is divided into 11 questions, with several sub questions in most of them, for a total of 36 questions. For the analysis, a 2 tailed Wilcoxon test was used. The results of the questionnaire are assembled into 8 scales and the average score for each scale before and after the treatment are compared. The alpha risk is 5%. The results are summarized on table 4 and the statistics on table 5.

TABLE 4
Means and standard deviations pre and post treatment for each scale

| | | Std. |
|---|---|---|
| | Mean | Deviation |
| PREPF Physical health | 47.78 | 25.51 |
| POSPF | 54.44 | 29.94 |
| PRERP Role physical | 25.00 | 12.50 |
| POSRP | 43.06 | 26.04 |
| PRERE Role emotional | 60.19 | 30.84 |
| POSRE | 81.48 | 20.32 |
| PRESF Social functioning | 25.93 | 16.67 |
| POSSF | 44.44 | 36.00 |
| PREBP Bodily pain | 27.00 | 17.68 |
| POSBP | 45.78 | 13.41 |
| PREGH General health | 43.22 | 14.10 |
| POSGH | 51.44 | 20.89 |
| PREVT Vitality | 21.11 | 11.40 |
| POSVT | 33.89 | 16.91 |
| PREMH Mental health | 56.44 | 7.33 |
| POSMH | 58.67 | 8.72 |
| PREHT1 Health transition | 3.11 | .33 |
| POSHT1 | 3.44 | 1.01 |

TABLE 5
Z scores for comparisons of the means and p values

| | | Asymp. Sig. |
|---|---|---|
| | Z | (2-tailed) |
| POSPF-PREPF Physical health | −.666(a) | .506 |
| POSRP-PRERP Role physical | −1.690(a) | .091 |
| POSRE-PRERE Role emotional | −1.859(a) | .063 |
| POSSF-PRESF Social | −1.552(a) | .121 |
| functioning | | |
| POSBP-PREBP Bodily pain | −1.997(a) | .046 |
| POSGH-PREGH General health | −1.548(a) | .122 |
| POSVT-PREVT Vitality | −2.198(a) | .028 |
| POSMH-PREMH Mental health | −.108(a) | .914 |
| POSHT1-PREHT1 Health | −1.134(a) | .257 |
| transition | | |

b Wilcoxon Signed Ranks Test

Interpretation: The results of this pilot study show that the product had significant beneficial effects on the subjects. Two scales, Bodily Pain and Vitality show differences with an alpha risk <5% the accepted threshold in sciences and 4 other with an alpha risk ≦12% which qualify them for “trend” because they are compatible with a true effect (and consequently have a good chance to be shown in a larger study).

It is noteworthy that among the 9 subjects tested, only 3 reported spectacular results such as the disappearance of a pain or more vitality or more hours of functioning. This suggests that the questionnaire can identify small improvements. The results of the comparison with the general population should be interpreted with caution because it is not a population matched for gender and age. They show however that each scale has shown a significant change for the better.

Conclusions and future direction: The results clearly support the hypothesis that the product has important beneficial effects on fatigue, pain and vitality. It also may have positive effects on mood. Finally, an observation on a possible satiety effect was made.

In the future, larger controlled and randomized trial should be designed to test the following hypothesis:

The product has beneficial effects on pain. Patients suffering from pain that are not adequately treated by conventional medicine, such as fibromyalgia may be a good population. Alternatively, patients who suffer from chronic pain which treatment may be harmful, such as arthritis could be considered.

The product has beneficial effects on fatigue. This could be tested on patients with chronic fatigue again.

In addition, pilot studies should test the following:

The product has beneficial effect on mood. An exploratory investigation with patients suffering from mood disorders or in women at menopause, who present with such problems, is indicated.

The product has satiety effects. This deserves a pilot study because if it is true, a huge portion of the population will be interested.

On a practical standpoint, the vehicle of the product should be worked on in order to obtain a palatable solution.

### Example 5

### Clinical Trial

### Product Summary

SKP is a new product for patents that suffer from Chronic Fatigue Syndrome (CFS) and Fibromyalgia (FMS) and that are coping with limitations due to pain, fatigue, and lack of energy and vitality.

SKP is a dried powder made of soy milk fermented with Kefir grains. The product has shown its efficacy in separate clinical trials on quality of life parameters such as bodily pain, role physical and vitality.

This presentation will summarize the results obtained from one of those clinical trials.

### Clinical Trial Design

The clinical trial was designed to confirm the results previously obtained by the Applicant by use of the SF-36v2 questionnaire.

Patients were to be recruited and divided in three treatment arms of 10 g, 20 g and 30 g per day of flavoured kefir powder.

Patients suffering from chronic fatigue syndrome and fibromyalgia of moderate intensity were selected. Symptoms of the disease include prominent fatigue as well as chronic widespread muscle and joint pain. These symptoms greatly limit a person's ability to work, entertain a social life, enter or maintain affective relationships, and practice any kind of physical activities.

Patients were followed for 60 days and SF36v2 questionnaires were filled at baseline, and after 30 and 60 days of treatment.

In the absence of a placebo group, statistical analysis aimed at comparing treatment groups at any time point to their baseline values by use of a two-tailed paired t-test.

### Clinical Trial Results

A total of 18 patients have been completed so far (10 at 20 g, 8 at 30 g). Approximately 9 patients are currently ongoing and should complete by mid-December.

There has been some drop-outs during the course of the study. Most patients dropped out of lack of compliance or lack of motivation (typical of CFS and FMS patients). Two patients stopped using the product for reasons of side effects in the 20 g group but still managed to finish the study. Those results were included in the analysis. Two patients also stopped the product on the 30 g group because of side effects. One of those two patients did not complete any follow-up questionnaire and was thus eliminated from the analysis.

Mean compliance was 55.6 days in the 20 g group Vs 49.8 days in the 30 g group.

The statistical analysis of the results of this clinical analysis is shown in FIGS. 4 to 9.

The results show a consistent and dose-dependent effect on many HRQOL parameters as summarized below in Table 6:

| HRQOL | | |
|---|---|---|
| parameter | Clinical meaningful | Statistical significance |
| Bodily Pain | All doses, all time points | 20 g @ 60 d, 30 g @ |
| | | 30, 60 d |
| Physical | 20 g @ 30, 60 d, 30 g @ 30 d | 20 g @ 60 d |
| Functioning | | |
| Role Physical | 20 g @ 60 d, 30 g @ 30, 60 d | 20 g @ 60 d |
| Social Function | 20 g @ 60 d, 30 g @ 30, 60 d | 20 g @ 60 d => |
| | | p = 0.064 |
| Vitality | 20 g @ 60 d, 30 g @ 30, 60 d | None |
| Role Emotional | 30 g @ 30, 60 d | None |
| Mental Health | 30 g @ 30, 60 d | None |
| General Health | None | None |

### REFERENCES

1. Zourari A, Anifantakis E M. Le kéfir: Caractères physicochimiques, microbiologiques et nutritionnels. Technologie de production. Une revue. Lait. 1988; 68:373-392.
2. Garrote G L, Abraham A G, De Antoni G L. Chemical and microbiological characterisation of kefir grains. J Dairy Res. 2001; 68:639-52.
3. Toba T, Arihara K., Adachi S. Distribution of microorganisms with particular reference to encapsulated bacteria in kefir grains. Int J Food microbiology. 1990; 10219-24.
4. Angulo L, Lopez E, Lema C. Microflora present in kefir grains of the Galician Region (North-West of Spain). J Dairy Res. 1993; 60:263-7.
5. Takizawa S, Kojima S, Tamura S, Fujinaga S, Benno Y, Nakase T. Lactobacillus kefirgranum sp. nov. and Lactobacillus parakefir sp. nov., Two New Species from Kefir Grains. Int J Syst Bacteriol. 1994; 44:435-9.
6. Cevikbas A, Yemni E, Ezzedenn F W, Yardimici T. Antitumoural, antibacterial and antifungal activities of kefir and kefir grain. Phytother Res. 1994; 8:78-82.
7. Lu K-M. Methods for inhibiting cancer growth, reducing infection and promoting general health with a fermented soy extract. US Patent Application No. 20020182274 filed Mar. 21, 2001.
8. Okamoto A, Hanagata H, Matsumoto E, Kawamura Y, Koizumi Y, Yanagida F. Angiotensin I converting enzyme inhibitory activities of various fermented foods. Biosci Biotechnol Biochem. 1995; 59:1147-9.
9. Chang, B W, Chen R L, Huang I J, Chang H C. Assays for angiotensin converting enzyme inhibitory activity. Ana. Biochem. 2001; 291:84-8.
10. Church F C, Swaisgood H E, Porter D H, Catignani G L. Spectrophotometric assay using ophthaldialdehyde for determination of proteolysis in milk and isolated milk proteins. J Dairy Sci. 1983; 66:1219-1227.
11. Hernandez-Ledesma, B, Amigo L, Ramos M, Recio I. Angiotensin converting enzyme inhibitory activity in commercial fermented products. Formation of peptides under simulated gastrointestinal digestion. J Agric Food Chem. 2004; 52:1504-10.
12. Shin Z, Yu R, Park S-A, Chung D K, Ahn C W, Nam H S, Kim K S, Lee H J. 2001. His-His-Leu, an angiotensin I converting enzyme inhibitory peptide derived from korean soybean paste, exerts antihypertensive activity in vivo. J Agric Food Chem. 2001; 49:3004-9.
13. Wu J, Ding X. Hypotensive and Physiological Effect of Angiotensin Converting Enzyme Inhibitory Peptides Derived from Soy Protein on Spontaneously Hypertensive Rats. J Agric Food Chem. 2001; 49:501-6.
14. Shin Z I, Ahn C W, Nam H S, Lee H J, Lee H J, Moon T H. Fractionation of angiotensin converting enzyme inhibitory peptide from soybean paste. Korean J Food Sci Technol. 1995; 27:230-4.
15. Adlercreutz H, Mazur W. Phyto-oestrogens and Western diseases. Ann Med. 1997; 29:95-120.
16. Fukutake M, Takahashi M, Ishida K, Kawamura H, Sugimura T, Wakabayashi K. Quantification of genistein and genistin in soybeans and soybean products. Food Chem Toxicol. 1996; 34:457-61.
17. Martin D S, Breitkopf N P, Eyster K M, Williams J L. Dietary soy exerts an antihypertensive effect in spontaneously hypertensive female rats. Am J Physiol Regul Integr Comp Physiol. 2001; 281:R553-60.
18. Martinez R M, Gimnénez I, Lou J M, Mayoral J A, Alda J O. Soy isoflavonoids exhibit in vitro biological activities of loop diuretics. Am J Clin Nutr. 1998; 68:1354s-1357s.
19. Nevala R, Korpela R, Vapaatalo H. Plant derived estrogens relax rat mesenteric artery in vitro. Life Sci. 1998; 63:95-100.
20. Gimenez I, Lou M, Vargas F, Alvarez-Guerra M, Mayoral J A, Martinez R M, Garay R P, Alda J O. Renal and vascular action of equol in the rat. J Hypertens 1997; 15:1303-8.
21. Mishra S K, Abbot S E, Choudhury Z, Cheng M, Khatab N, Maycock N J, Zavery A, Aaronson P I. Endothelium-dependent relaxation of rat aorta and main pulmonary artery by the phytoestrogens genistein and daidzein. Cardiovasc Res. 2000; 46:539-46.
22. Duarte J, Perez-Palencia R, Vargas F, Ocete M A, Perez-Vizcaino F, Zarzuelo A, Tamargo J. Antihypertensive effects of the flavonoid quercetin in spontaneously hypertensive rats. Br J Pharmacol. 2001; 133:117-24.
23. Sadowska-Krowicka H, Mannick E E, Oliver P D, Sandoval M, Zhang X J, Eloby-Childess S, Clark D A, Miller M J. Genistein and gut inflammation: role of nitric oxide. Proc Soc Exp Biol Medicine. 1998; 217:351-7.
24. Kong L Y, Lai C, Wilson B C, Simpson J N, Hong J S. Protein tyrosine kinase inhibitors decrease lipopolysaccharide-induced proinflammatory cytokine production in mixed glia, microglia-enriched or astrocyte-enriched cultures. Neurochem Int. 1997; 30:491-7.
25. Deodato B, Altavilla D, Giovanni S. Cardioprotection by the phytoestrogen genistein in experimental myocardial ischemia-reperfusion injury. Br J. Pharmacol. 1999; 128:1683-90.
26. Regal J F, Fraser D G, Weeks C E, Greenberg N A. Dietary Phytoestrogens Have Anti-Inflammatory Activity in a Guinea Pig Model of Asthma. Proc Soc Exp Biol Med. 2000; 223:372-8.
27. Guven A, Guven A, Gulmez M. The effect of kefir on the activities of GSH-Px, GST, CAT, GSH and LPO levels in carbon tetrachloride-induced mice tissues. J Vet Med B Infect Dis Vet Public Health. 2003; 50:412-6.
28. Kubow S, Foutouhinia M. Kefir as a potent antioxidant composition. U.S. Patent Ser. No. 60/213,268 filed Jun. 22, 2000. International Patent Application No. PCT/CA01/00899 filed Jun. 18, 2001.
29. Alm L. Survival rate of Salmonella and Shigella in fermented milk products with and without added human gastric juices: An in vitro study. Prog Food Nutr Sci. 1983; 7:19-26.
30. Korneva V V, Nabukhotnyi T K, Cherevko S A, Kravets A A, Sidorchuk I I. Use of propiono-acidophylus milk in the complex treatment of intestinal disbacteriosis in infants with taphylococcal infections and sepsis. Vopr Pitan. 1979; 5:37-40.
31. Morgan S M, Hickey R, Ross R P, Hill C. Efficient method for the detection of microbially-produced antibacterial substances from food systems. J Appl Microbiol. 2000; 89:56-62.
32. Batinkov E L. Use of milk and kefir in peptic ulcer of the stomach and duodenum. Vopr Pitan. 1971; 30:89-91.
33. Murofushi M, Shiomi M, Aibara K. Effect of orally administered polysaccharide from kefir grain on delayed-type hypersensitivity and tumor growth in mice. Jpn J Med Sci Biol. 1983; 36:49-53.
34. Osada K, Nagira K, Teruya K, Tachibana H, Shirahata S, Murakami H. Enhancement of interferon-b production with sphingomyelin from fermented milk. Biother. 1994; 7:115-123.
35. Thoreux K, Schmucker D L. Kefir milk enhances intestinal immunity in young but not old rats. J. Nutr. 2001; 131:807-12.
36. Kubow S, Chan H M, Chen C, Fotouhinia M. Kefir extract as an anti-cancer agent. U.S. Patent Ser. No. 60/211,804 filed Jun. 16, 2000. International Patent Application No. PCT/CA01/00896 filed Jun. 15, 2001.
37. Liu J R, Wang S Y, Lin Y Y, Lin C W. Antitumor activity of milk kefir and soy milk kefir in tumor-bearing mice. Nutr Cancer. 2002; 44:182-187.
38. Murofushi M, Mizuguchi J, Aibara K, Matuhasi T. Immunopotentiative effect of polysaccharide from kefir grain, KGF-C, administered orally in mice. Immunopharmacology 1986; 121:29-35.
39. Kubo M, Odani T, Nakamura S, Tokumaru S, Matsuda H. Pharmacological study on Kefir—a fermented milk product in Caucasus. I. On antitumor activity. Yakugaku Zasshi. 1992; 112:489-95.
40. Shiomi M, Sasaki K, Murofushi M, Aibara K. Antitumor activity in mice of orally administered polysaccharide from Kefir grain. Jpn J Med Sci Biol. 1982; 35:75-80.
41. Shiomi M, Aibara K, Murofushi M. Effect of orally administered polysaccharide from kefir grain on delayed-type hypersensitivity and tumor growth in mice. Jpn J Med Sci Biol. 1983; 36:49-53.
42. Astwood J D, Leach J N, Fuchs R L. Stability of food allergens to digestion in vitro. Nat Biotechnol. 1996; 14:1269-73.
43. Kiers J L, Van Laeken A E, Rombouts F M, Nout M J. In vitro digestibility of bacillus fermented soya bean. Int J Food Microbiol. 2000; 60:163-9.
44. Roberts P R, Burney J D, Black K W, Zaloga G P. Effect of chain length on absorption of biologically active peptides from the gastointestinal tract. Digestion. 1999; 26:222-227.
45. Masuda O, Nakamura Y, Takano T. Antihypertensive peptides are present in aorta after oral administration of sour milk containing these peptides to spontaneously hypertensive rats. J. Nutr. 1996; 126:3063-8.
46. Manach C, Scalbert A, Morand M, Remesy C, Jimenez L. Polyphenols: food sources and bioavailability. Amer J Clin Nutr. 2004; 79:727-47.
47. Kinoshita E, Yamakoshi J, Kikuchi M. Purification and Identification of an Angiotensin I-Converting Enzyme Inhibitor from Soy Sauce. Biosci Biotechnol Biochem. 1993; 57:1107-10.
48. Fitzgerald R J, Murray B A, Walsh D J. Hypotensive peptides from milk proteins. J. Nutr. 2004; 134: 980S-988S.
49. Blomstrand E, Hassmen P, E k S, et al. Influence of ingesting a solution of branched-chain amino acids on perceived exertion during exercise. Acta Physiol Scand 1997; 159:41-9.
50. De Lorenzo A, Petroni M L, Masala S, et al. Effect of acute and chronic branched-chain amino acids on energy metabolism and muscle performance. Diabetes Nutr Metab. October 2003; 16(5-6):291-7.
51. Faipoux R, Tome D, Bensaid A, Morens C, Oriol E, Bonnano L M, Fromentin G. Yeast proteins enhance satiety in rats. J Nutr. 2006 September; 136(9):2350-6.)
52. Georgiades E, Behan W M, Kilduff L P, Hadjicharalambous M, Mackie E E, Wilson J, Ward S A, Pitsiladis Y P. Chronic fatigue syndrome: new evidence for a central fatigue disorder. Clin Sci (Lond). 105:213-8, 2003.
53. Lieberman J and Bell D S: Serum angiotensin-converting enzyme as a marker for the chronic fatigue-immune dysfunction syndrome: A comparison to serum angiotensin-converting enzyme in sarcoidosis. Am Med J Med 95, 407-412, 1993.
54. Lund T D, West T W, Tian L Y, Bu L H, Simmons D L, Setchell K D, Adlercreutz H, Lephart E D. Visual spatial memory is enhanced in female rats (but inhibited in males) by dietary soy phytoestrogens. BMC Neurosci 2001a; 2:20.
55. Lund T D, Lephart E D. Dietary soy phytoestrogens produce anxiolytic effects in the elevated plus-maze. Brain Res 2001; 913:180-184.
56. Nissen, S. L., R. Sharp, M. Ray, J. A. Rathmacher, D. Rice, J. C. Fuller, Jr., A. S. Connelly, and N. Abumrad. Effect of leucine metabolite hydroxy-b-methylbutyrate on muscle metabolism during resistance-exercise training. J. Appl. Physiol. 81:2095-2104, 1996.
57. Rowbotham M C. What is a “clinically meaningful” reduction in pain? Pain 2001. 94:131-132.
58. Simon N G, Kaplan J R, Hu S, Register T C, Adams M R. Increased aggressive behavior and decreased affiliative behavior in adult male monkeys after long-term consumption of diets rich in soy protein and isoflavones. Horm Behav 2004; 45:278-284.

1. A method for treating fatigue and/or mood disorders and/or improving satiety and/or vitality in a subject in need thereof, comprising the step of administrating an effective amount of a soy kefir powder to said subject.

2. The method of claim 1 wherein the soy kefir powder is obtained by fermentating soymilk with kefir grains from the Moscow kefir strain in a ratio of 1 part grains to 40 parts soymilk, to obtain a fermentation culture wherein the kefir grains are separated from the fermentation culture to obtain a fermentation liquid and wherein the fermentation liquid is then spray-dried to form a soy kefir powder.

3. The method of claim 1 wherein the soy kefir powder has at least one of the following characteristics:
a protein composition of approximately of 25-45%,
a carbohydrate composition of approximately of 5-45%,
a fat composition of approximately of 25-45%, or
an ash composition of approximately of 5-15%.

4. The method of claim 1, wherein the soy kefir powder has at least one of the following characteristics:
a protein composition of 35-40% wet weight,
a carbohydrate composition of 10-14% wet weight,
a fat composition of 35-40% wet weight,
a moisture of 8-10% wet weight,
an ash composition of 5-7% wet weight, or
a pH of 4.2.

5. The method of claim 1, wherein the soy kefir powder has at least three of the following characteristics:
a protein composition of 35-40%,
a carbohydrate composition of 10-14%,
a fat composition of 35-40%,
a moisture of 8-10%,
an ash composition of 5-7%, or
a pH of 4.2.

6. The method of claim 1, wherein the soy kefir powder has the following characteristics:
a protein composition of 35-40%,
a carbohydrate composition of 10-14%,
a fat composition of 35-40%,
a moisture of 8-10%,
an ash composition of 5-7%, and
a pH of 4.2.

7. The method of claim 1, wherein the soy kefir powder is associated with an acceptable carrier.

8. The method of claim 1 wherein the acceptable carrier is a liquid.

9. The method of claim 1, wherein the liquid acceptable carrier is water, milk, juice.

10. The method of claim 1 wherein the acceptable carrier is a yogourt.
